(19) United States
(12) Patent Application Publication
(10) Pub. No.: US 2014/0038225 A1
(43) Pub. Date: Feb. 6, 2014

(54) CELL CULTURE DEVICE

(71) Applicants: Nils Goedecke, Zürich (CH); Ralf Streichan, Zürich (CH)

(72) Inventors: Nils Goedecke, Zürich (CH); Ralf Streichan, Zürich (CH)

(21) Appl. No.: 14/110,692

(22) PCT Filed: Mar. 30, 2012
(86) PCT No.: PCT/EP12/01429

§ 371 Date: Oct. 8, 2013

(30) Foreign Application Priority Data
Apr. 12, 2011 (EP) 11 003 086.3

(51) Int. Cl. C12Q 1/02

(52) U.S. Cl. CPC C12Q 1/025
USPC 435/29; 435/288.3; 73/862.541

(57) ABSTRACT

A cell culture device for culturing cells and measuring mechanical forces exerted by one or more cells. The device includes a cell culture chamber having a bottom plate and a main frame with side walls to enclose a volume. The main frame has side walls fabricated as one piece. The cell culture chamber is open on a side opposite the bottom plate. The device further has a force sensor array on top of the bottom plate within the cell culture chamber. The force sensor array has an array of flexible microcolumns configured to provide a site of adhesion for one or more cells, each microcolumn being configured to be deflected from an original position to a deflected position in response to a mechanical force exerted by the one or more cells.

The present invention relates to a cell culture device for culturing cells and measuring mechanical forces exerted by one or more cells, comprising a cell culture chamber and a force sensor array. The present invention further relates to a force sensor array for use as part of the cell culture device according to the present invention, and a lid for use with the cell culture device according to the present invention. Under a further aspect, the present invention relates to a method of culturing one or more cells and measuring mechanical forces exerted by the one or more cells in the device according to the present invention.

Many different cell types are capable of exerting forces to their surrounding environment. Most prominent for such phenomena are muscle cells. Muscle cells can be subdivided into: i) smooth muscle cells forming vascular structures, ii) cardiac muscle cells forming cardiac muscles and iii) skeletal muscle cells forming muscle fibres. However, there are many other cells types that show contraction or relaxation, e.g. fibroblasts in connective tissue or bronchial cells in lung tissue. Also carcinoma cells can exert forces on the surrounding cells helping them migrate through tissue.

Studies on cell contraction, relaxation or migration are performed for a better understanding of physiological processes that influence cell adhesion and for extending principal knowledge on cytomechanics. Forces exerted by cells (e.g. cell contraction, cell relaxation and cell migration) have been investigated for more than 50 years. Still, there is a vast interest in this topic, particularly with upcoming new materials and research tools.

Investigations on cell contraction are routinely performed by physiologists and pharmacologists. Traditionally, such studies are utilising whole tissue in myograph-organ bath setups or cell layers in gel matrices. Recently, cultivating cells on top of elastic microposts, also called microneedles or micropillars, has become a technique employed by such scientists. These often cylindrical structures are force sensors, which are typically arranged in arrays. These arrays are advantageous in that they allow measuring forces exerted by cells to a surface with a substantially higher resolution in force measurement than planar gel structures. Another advantage of micropost assays over gel structures is the ability to study relaxation. An example of such a micropost array can be found in John L. Tan et al., “Cells lying on a bed of microneedles: An approach to isolate mechanical force”, PNAS, Vol. 100, No. 4, pp. 1484-1489 (February 2003). So far, cells are cultured in a first step, then transferred to a device where mechanical forces are measured in a separate step.

Therefore, there exists a need for a device that would enable both cell culture and measurement of mechanical forces exerted by the cultured cells in one and the same device without the need to provide additional complex structures. Ideally, the device would be disposable, i.e. for single use.

In a first aspect, the present invention provides a cell culture device for culturing cells and detecting mechanical forces exerted by one or more cells, comprising: a cell culture chamber, comprising a bottom plate and a main frame comprising side walls to enclose a volume of the culture chamber together with the bottom plate, the main frame with side walls being fabricated as one piece, the cell culture chamber being open on a side of the cell culture chamber opposite the bottom plate, the cell culture chamber further comprising a force sensor array disposed on top of the bottom plate within the cell culture chamber, the force sensor array comprising an array of flexible microcolumns configured to provide a site of adhesion for one or more cells, each microcolumn being configured to be deflected from an original position to a deflected position in response to a mechanical force exerted by the one or more cells.

The device according to the present invention is configured to allow cell culture first and then, without the need to transfer cells or transfer the container containing the cells to a separate apparatus, detect mechanical forces exerted by the cells in one and the same device.

Detection of mechanical forces may be of a qualitative nature only, or may comprise measurement of mechanical forces exerted by the cells, in particular quantitative measurement. In a preferred embodiment, detecting mechanical forces entails measuring mechanical forces.

The force sensor array is provided as part of the cell culture device. In particular, the force sensor array is fixedly attached to the cell culture device. It may be directly attached to the bottom plate, or it may be attached via one or more intermediate layers or structures to the bottom plate. The device is configured such that it allows for growing cells inside the cell culture chamber on top and next to the force sensor array. The device of the present invention is advantageous in that its handling is simple, not only for an expert in the field. In addition, it does not require any additional tools other than standard in cell culture. Since the cells do not have to be transferred and/or inserted into a separate flow chamber, measuring chamber or the like, they do not get bruised and the potential to get artefacts from unwanted cell death is eliminated.

In exemplary embodiments of the present invention, at least a part of the bottom plate is provided by a transparent plate, or else, at least a part of the bottom plate is transparent. In a further embodiment, the bottom plate is a transparent plate. Preferably also, the force sensor array is transparent and arranged on top of a transparent section of the bottom plate, rendering the assembly of force sensor array on top of the bottom plate transparent. That way, optical inspection of cells and detection of mechanical forces is possible from either side of the cell culture device.

Most preferably, the bottom plate is made from a rigid, i.e. substantially inflexible material. In an exemplary embodiment, the transparent plate is a glass plate. This glass plate may, for example, be a simple microscope slide or cover slide.

The main frame may comprise a holding structure for holding the bottom plate. According to one exemplary embodiment, the holding structure may simply comprise or consist of a flat section surrounding the side walls, such as a flat plate, preferably at least outside an area enclosed by the side walls. In other words, the flat section may be provided by a flat plate having an opening inside the area enclosed by the side walls. The bottom plate would then preferably be arranged at least in the area of the opening. This flat section (flat plate) can provide a flat surface running parallel to a surface of the bottom plate, to which the bottom plate may be fixed. In the alternative, the holding structure may comprise a flat section having an opening or at least a recessed section of a size that is adapted to surround the bottom plate on all sides, preferably tightly surround the bottom plate on all sides. This opening or recessed section may be designed to provide such a tight fit for the bottom plate that the bottom plate can be inserted into the opening or recessed section and be held therein simply by friction. In the alternative, the recessed section may, in turn, comprise a flat section surface running parallel to a surface of the bottom plate, to which the bottom plate may be fixed. In those embodiments, the recessed section preferably has a depth, as measured relative to the surface from the section is recessed, which matches the thickness of the bottom plate inserted into the recess, such that a surface spanning the flat surface comprising the recess and the bottom plate is substantially even.

The bottom plate may be fixed to the main frame in a variety of ways, such as by clamping or other mechanical means, in particular means that allow releasing the bottom plate again. However, preferably, the bottom plate is permanently attached to the main frame. This can be effected by gluing, welding or bonding, for instance. Welding may involve ultrasonic welding, for example.

In other exemplary embodiments, the bottom plate is an integral part of the main frame, i.e. is formed as one piece with the main frame. In such embodiments there is no need for an extra step of fixing a bottom plate to the main frame, thus saving production time and cost.

In preferred embodiments, the main frame is made from a polymeric material, preferably a transparent polymeric material. In further preferred embodiments, the main frame is fabricated by injection moulding. The use of polymeric material for the main frame, in particular in combination with injection moulding, allows for cost-efficient mass production.

As a result, the device according to the invention is preferably a disposable device. Disposable devices may be discarded after a single use. Such a completely disposable system allows for sterile cell culture without the risk of cross contamination from earlier experiments.

The main frame and/or the bottom plate may be made of a carbon-based polymer, for instance. By way of example the main frame and/or the bottom plate may be made of polycarbonate, polymethacrylate, polystyrene, cellulose acetate based polymer or like materials. In the alternative, the main frame and/or the bottom plate may be made of a silicone-based polymer, for instance of a poly(organo)siloxane.

The bottom plate may have a thickness of a cover slide. The bottom plate preferably has a thickness in the range of between 0.05 to 0.5 mm, for instance 0.07 to 0.25 mm.

The sidewalls may have a height of between 1 and 50 mm, preferably 2 to 30 mm, and more preferably 5 to 20 mm and/or a thickness of between 0.1 and 5 mm, preferably 0.2 to 3 mm, and more preferably 0.5 to 2 mm. The height designates the distance between the two planes defined by the openings on either side in the cell culture chamber (with one opening being sealed by the bottom plate), the thickness designating a dimension in the plane defined by the opening. The cell culture chamber may have a width of between 2 to 50 mm, for instance, preferably 3 to 30 mm and more preferably 5 to 20 mm.

The cell culture chamber may have a volume in the range of from 0.01 to 10 millilitres, for instance, preferably 0.05 to 5 millilitres, more preferably from 0.1 to 3 millilitres.

The opening on one side of the cell culture chamber allows for easy access (from top). This allows using a manual pipette or a robotic dispenser for dispensing or exchanging the fluid inside. Furthermore, an open cell culture device allows performing confocal microscopy using dipping lenses.

In an exemplary embodiment of the present invention, the cell culture device comprises two or more culture chambers. The main frame would then provide a common platform for all culture chambers. The cell culture device may have any desired format. For instance, the cell culture device may have a size of about 76 mm×26 mm. In other embodiments, the cell culture device may have the size of a standard 96-well plate with 96 cell culture chambers and/or 96 observation spots being arranged substantially in the same manner as the 96 wells in the 96-well plate. An observation spot is an array of a number of microcolumns sufficient for the detection of the mechanical forces exerted by one or a few cells. A force sensor array comprises at least one observation spot, typically a plurality of observation spots.

The force sensor array is disposed on top of the bottom plate within the cell culture chamber. It comprises an array of flexible microcolumns configured to provide a site of adhesion for one or more cells, each microcolumn being configured to be deflected from an original position to a deflected position in response to a mechanical force exerted by the one or more cells. As will be described in more detail further below, the force sensor array preferably comprises a plurality of discrete observation spots, which are spaced apart from one another, each observation spot comprising a plurality of flexible microcolumns Force sensor arrays as such have been known from the prior art, as mentioned before. The principle these force sensor arrays are based on is briefly described in the following with reference to FIG. 1.

FIG. 1 shows a section of a cell culture device respectively cell culture chamber in accordance with the present invention. The top part of FIG. 1 shows a cross-section (as seen from the side). The lower part of FIG. 1 shows a top view onto an observation spot of a force sensor array. The observation spot is disposed on top of bottom plate 15, which may be a glass cover plate, for instance. The observation spot comprises a supporting base foil 14 with a plurality of microcolumns 13 disposed thereon. Typically, the microcolumns and the supporting base foil are manufactured as one piece and may be made of PDMS, for instance. On the left-hand side of FIG. 1, a cell 11 is shown which spreads over and adheres to plural microcolumns, more particularly the tops thereof. At that stage, the microcolumns are still arranged in the original regular pattern, with the original positions of the microcolumns being shown by reference numeral 19. A stimulus, such as a chemical stimulus as symbolized by droplet 16, triggers a cell reaction, the cell may contract or relax. Contracted cell 12 then deflects the microlumns away from their original positions 19 into deflected positions 18, as apparent from the right-hand side of FIG. 1. The deflection from the original to the deflected positions can be represented by force vectors 17. Thus, each microcolumn is a force sensor. As described by Tan et al., for small deflections, the microcolumns behave like springs such that the deflection is directly proportional to the force applied by the cell. For further details of the mathematical description of the deflections and measurement of the mechanical forces exerted by the cell, it is referred to John L. Tan et al., “Cells lying on a bed of microneedles: An approach to isolate mechanical force”, PNAS, Vol. 100, No. 4, pp. 1484-1489 (February 2003), which is incorporated in its entirety by reference herein.

Each or at least a plurality of the microcolumns of the force sensor array may, for instance, have a substantially circular, substantially elliptical, substantially rectangular, substantially rhombic or substantially square shape, when seen from the top (i.e. perpendicular to the bottom plate on which the force sensor array is disposed). The microlumns may all have the same or a different shape. Preferably, at least the microcolumns within an observation spot have the same shape.

The choice of material, side length or diameter and height of the microlumns determines the stiffness of the microcolumns and can be tailored to meet the demands posed by the particular application and thus forces exerted by the one or more cells.

Each of the microcolumns of the force sensor array can have a height of between 0.5 and 50 micrometers, for instance between 1 and 45 micrometers, such as between 2 and 40 micrometers.

In one exemplary embodiment, each of the microcolumns in one observation spot, or each of the microcolumns of the force sensor array has a substantially circular shape with a diameter in the range of from 0.5 to 50 micrometers. In exemplary embodiments, the diameter is in the range of between 0.75 and 40 micrometers, and may be between 1 and 25 micrometers. In further exemplary embodiments, the diameter would be between 2 and 12 micrometers.

In one exemplary embodiment, each of the microcolumns in one observation spot, or each of the microcolumns of the force sensor array has a square, rhombic or rectangular shape characterized by side lengths in the range of from 0.5 to 50 micrometers. In exemplary embodiments, the side lengths are in the range of between 0.75 and 40 micrometers, and may be between 1 and 25 micrometers. In further exemplary embodiments, the side lengths would be between 2 and 12 micrometers. The microcolumns within one observation spot may be the same in terms of geometry and/or dimensions and/or stiffness or differ from one another in terms of geometry and/or dimensions and thus stiffness. In addition, the microcolumns in an observation spot may be evenly spaced apart or may be arranged in a pattern, which may be regular or irregular, depending on the particular application. For instance, an observation spot may comprise a first area wherein microcolumns have a first stiffness and/or geometry and are arranged at a first spacing and at least a second area wherein the microcolumns have a second stiffness and/or geometry and are arranged at a second spacing, wherein the first and second stiffness and/or geometry and/or the first and second spacing are different from one another. In one exemplary embodiment, the microcolumns in one observation spot are evenly spaced apart and/or have the same stiffness, in particular geometry and dimensions. In further embodiments, the microcolumns in one observation spot may be different in stiffness, for instance different in geometry, dimensions and/or spacing, from the microcolumns in another observation spot. For instance, the force sensor array may comprise a plurality of observation spots wherein the stiffness and/or spacing (density) of the microcolumns in one observation spot differs from the stiffness and/or spacing (density) of the microcolumns in an adjacent observation spot. Including observation spots, which differ from one another in terms of the spacing and/or geometry and/or stiffness of the microcolumns is advantageous in that it provides favourable conditions for different cell types. Different cell types prefer different microcolumn arrangements, for instance different spacings between the microcolumns (microcolumn density). Also, cells may react in different ways to different microcolumn arrangements, which reactions allow valuable insights into cell behaviour. For instance, the formation of anchoring points or antennae may differ depending on the environment. Cancer cells move or may be made to move on the observation spots. If the observation spot comprises areas that differ in terms of the arrangement and/or stiffness and/or geometry of the microcolumns, the direction and speed of movement of the cancer cells can provide valuable information, for instance about their cytoskeleton.

The microcolumns of the force sensor array, in particular at least the microcolumns in one observation spot, may be spaced apart by a distance in the range of from 1 to 50 micrometers, for instance by a distance in the range of from 5 to 45 micrometers, or by a distance between 10 and 40 micrometers. The distance is measured from one centre of a microcolumn to the centre of the nearest microcolumn.

Typically, the microcolumns are integrally formed with a supporting base foil. The supporting base foil may suitably have a thickness of between 0.05 and 2 millimetres, for instance between 0.5 and 1 millimetres. The supporting base foil is then disposed on top of and typically in parallel with the bottom plate of the cell culture chamber. That way, the overall thickness of the sensor array meets the working distance criteria not only for long distance lenses but also for many immersion lenses.

The force sensor array may be made from a variety of suitable transparent materials, such as a silicone elastomer, a mixture of silicone elastomers or a mixture of a silicone elastomer and a silicone fluid, for instance. The transparent silicone elastomer is, for instance, preferably polydimethylsiloxane (PDMS). A suitable PDMS material is known under the trade name Sylgard®, for instance. Considering the Young's modulus of silicone elastomers, the spring constant of such a microcolumn is in the range of a few ten Nano-Newtons for each micrometer displacement. The material of the force sensor array and/or the cell culture device may include one or more compounds for changing the property of the (neat) material, such as compounds providing colour and/or magnetic properties to the material. Other functional compounds may be used to change the refractive index and/or conductivity of the material. Expressed differently, the material may include a colourant, an electrically conductive or insulating material, a magnetic compound and/or a compound having a refractive index different from the refractive index of the material.

The force sensor array can be made by conventional methods as known in the art. For instance, the force sensor array can be made by producing a negative pattern of the array by standard lithographic methods. Liquid prepolymer of PDMS, for instance, is then used to fill the negative pattern. In those embodiments where a glass plate is used as a bottom plate, the glass plate can be activated by plasma treatment and placed onto the liquid prepolymer of PDMS, thus providing a bond between bottom plate and the surface of the PDMS, once it is cured, which will later become the supporting base foil. Another way of fabrication is the use of replica-moulding, involving preparation of a template comprising an array of holes in PDMS from a silicon template comprising microcolumns. Manufacture of the microcolumns then involves filling the template comprising the array of holes with PDMS prepolymer, curing and peeling off the template.

In order to promote adhesion of cells to the microcolumns, the microcolumns are preferably at least partially treated. Suitable treatments include coating with fibronectine, laminine or collagen, for instance by immersion of the force sensor array into a suitable solution or microcontact printing onto at least a part of the microcolumns.

In order to suppress adhesion of cells to the side of the microcolumns, the microcolumns are preferably at least partially treated. Suitable treatments include coating with Pluronic 127 solution or bovine serum albumin (BSA), for instance by immersion of the force sensor array into a suitable solution or microcontact printing onto at least a part of the microcolumns.

In exemplary embodiments of the present invention, the cell culture device further comprises a perfusion system. The perfusion system serves to deliver liquid to and remove liquid from the cell culture chamber, or possibly plural cell culture chambers, if the cell culture device comprises more than one cell culture chamber. The liquid may be cell culture medium, for instance. The liquid may also be a chemical stimulant, for instance.

The perfusion system preferably forms an integral part of the main frame, i.e. at least a part or all of its structure is preferably formed as a component of the main frame. In exemplary embodiments, the perfusion system comprises at least one microfluidic channel providing fluidic communication between a connector located on the main frame outside the cell culture chamber and the cell culture chamber. The perfusion system may comprise two or more microfluidic channels and optionally connectors.

For example, the perfusion system can comprise a first microfluidic channel providing fluidic communication between a first connector located on the main frame outside the cell culture chamber and the cell culture chamber for delivering fluid into the cell culture chamber, and a second, separate microfluidic channel providing fluidic communication between a second connector located on the main frame outside the cell culture chamber and the cell culture chamber for removing fluid from the cell culture chamber.

The first and/or second connectors may be provided in any suitable format. The simplest form would be just an opening in the surface of the main frame. Advantageously, the first and/or second connectors are provided in a form that allows simple interfacing to fluid delivery or removal systems, for instance in the form of tubes, in particular round tubes as common in the art, extending from the surface of the main frame in the same direction as the side walls of the cell culture chamber(s). Suitable fluid delivery or removal systems may be vacuum pumps, peristaltic pumps, syringe pumps, or delivery systems based on hydrostatic pressure that may be connected to the connectors via conventional tubing, to name but a few. In a further conceivable embodiment, the first connector has sufficient volume relatively above the bottom plate of the cell culture chamber that it may serve as a reservoir from which fluid is driven by hydrostatic pressure into the cell culture chamber. These considerations apply equally to any connectors that may be provided in addition to the first and/or second connectors.

In order to facilitate handling of the perfusion system, the first and/or second connectors may have indicators associated therewith to identify their functions as inlet (delivery) and outlet (removal). The indicators may be of the same or a different kind and may be one or more letters, symbols, such as arrows, numbers or the like.

In addition, in particular the first microfluidic channel may comprise a microfluidic channel system comprising more than one microfluidic channel section, optionally in combination with more than one connector. The first microfluidic channel may comprise a microfluidic channel system comprising plural interconnected microfluidic channel sections. For instance, the first microfluidic channel system may comprise three microfluidic channel sections connected by a T- or Y-junction, with one microfluidic channel section leading into the cell culture chamber, as described before, a further microfluidic channel section being connected to the first connector and a further microfluidic channel section being connected to a further connector. Such a configuration allows mixing one or more fluids during their passage into the cell culture chamber, for instance. It would also allow reaction of components comprised in the fluid. Furthermore, such an arrangement enables a fast switching between two or more different culture media.

The second microfluidic channel may be connected to the cell culture chamber by an opening disposed at a predetermined distance of at least 100 micrometers, preferably at least 200 micrometers, at least 500 micrometers or at least 1 mm from the bottom plate of the cell culture chamber within the cell culture chamber, measured perpendicular to the bottom plate of the cell culture chamber. The opening is preferably disposed such that when operating the device, any fluid in excess of a predetermined fill volume will flow into the opening and exit from the cell culture chamber. Thus, such an arrangement serves as an overflow system, in which excess fluid in the cell culture chamber would automatically drain from the cell culture chamber, thus greatly facilitating filling the cell culture chamber with predetermined volumes of liquid and/or maintaining a constant fill level of liquid in the cell culture chamber, respectively.

Such an arrangement is, of course, equally conceivable in case of one microfluidic channel per cell culture chamber only, in which case it would concern the first (and only) microfluidic channel. In a further conceivable embodiment, a plurality of such second microfluidic channels with corresponding openings may be provided within a cell culture chamber.

This aspect, respectively feature, of the present invention may serve as an integrated liquid level regulation that is part of the microfluidic perfusion. Liquid can be easily removed from the cell culture chamber. This opening may therefore also be referred to as fill level overflow opening in the following. In those embodiments where the cell culture chamber is closed to maintain a controlled atmosphere within the cell culture chamber, this is particularly advantageous.

In an alternative embodiment, an opening connecting the second connector to the second microfluidic channel may be disposed at any suitable position within the cell culture chamber. Providing such a perfusion system does not only allow exchange of culture medium and supply of chemical stimulants, it also allows to provide defined shear conditions.

In those embodiments providing first and second microfluidic channels and thus a fluid inlet and outlet, a ratio between controlled influx from the inlet (first connector) and suction at the outlet (second connector), for instance, allows keeping a steady flow through the chamber. This enables to grow cells under defined shear conditions or to perform contraction experiments in an automated fashion.

In one exemplary embodiment, the opening (fill level overflow opening) is disposed in a sidewall or adjacent to a sidewall in the cell culture chamber. In terms of ease of manufacturing and in terms of mechanical stability, it may be advantageous if the opening is formed integrally with a sidewall, i.e. provided as one piece with the sidewall.

In other conceivable embodiments, the opening may be provided as a stand-alone-feature within the cell culture chamber, for instance provided by a tube.

Preferably, the opening has a funnel-shape, thus facilitating entry of liquid into the opening.

The main frame may, for instance, comprise a flat plate carrying the side walls and the connector, with the microfluidic channel or microfluidic channels being disposed on a side of the main frame opposite the side walls and the connector, i.e. a bottom side of the main frame. The microfluidic channel(s) may then be sealed on one side by the bottom plate.

The at least one microfluidic channels can have a width of 0.05 mm to 5 mm and/or a height of 0.05 mm to 5 mm, for instance. Widths and/or heights of less than 1 mm, preferably less than 0.5 mm are preferred, however.

According to a further aspect of the present invention, a cell culture device as described herein and in claim 1, comprises the above described perfusion system, but does not comprise a force sensor array.

As set out before, one side (the top side in use) of the cell culture chamber is open. In an exemplary embodiment according to the present invention, the cell culture device further comprises a lid for closing the cell culture chamber.

In those embodiments wherein the cell culture device comprises more than one cell culture chamber, it may comprise a lid for each cell culture chamber, or may comprise a common lid suitable to simultaneously close the more than one cell culture chambers. In the latter case, the common lid may comprise an array of interconnected lids for each of the more than one cell culture chambers.

The lid may advantageously comprise a structural feature providing for two closing modes: a first closing modus wherein the lid provides an air-tight seal to the side walls and a second closing modus providing a gap between the lid and the side walls, or providing an opening through both a section of the lid and side wall, allowing gas exchange between the cell culture chamber and the environment outside the cell culture chamber. The structural feature may be a spacer.

Typically, in the first closing modus, the lid is applied to the sidewalls in a first position, and in the second closing modus, the lid is applied to the sidewalls in a second position, which is different from the first position. In other words, different lid positions enable two modes for controlled gas exchange, including no gas exchange.

In exemplary embodiments, the cell culture chamber or the main frame comprises a corresponding structural feature such that when the lid is placed on the cell culture chamber in a first position, the structural features come in contact or engage otherwise in the second closing modus, and when the lid is placed on the cell culture chamber in a second position, which is different from the first position, the structural features do not come in contact and do not engage, thus providing the first closing modus. For instance, in case of rectangular cell culture chambers, the first position may be a 90°-turn with regard to the second position or vice versa.

The main frame may comprise a structural feature configured to supplement the structural feature provided on the lid such that the lid comes to rest in the first closing position. For instance, a spacer provided on the lid may be received in a corresponding, spacer-receiving section (recess formed in the shape of the spacer, for instance). This structural feature may be disposed on a sidewall of the cell culture chamber or adjacent to the sidewall, or it may be a stand-alone structural feature.

The structural feature on a side wall of the cell culture chamber may, for instance, be an indentation or through-hole in a rim provided around the side walls of the cell culture chamber and the structural feature provided on the lid may then be a protrusion configured to fit into the indentation or through-hole.

The structural feature on a side wall of the cell culture chamber may be a protrusion and the structural feature provided on the lid may an indentation or recess configured to receive the protrusion such that the lid comes to rest in the first closing position, for instance.

The lid may further comprise one or more symbols or other indicators indicating a first and/or second position of the lid relative to the cell culture chamber(s). In addition or alternatively, the main frame may further comprise one or more symbols or indicators to indicate a first and/or second position of the lid relative to the cell culture chamber(s).

A further aspect of the present invention concerns the lid as described above as such. Yet a further aspect of the present invention concerns a cell culture device comprising the lid as described above, but comprising no force sensor array.

The cell culture device according to the present invention may thus comprise a perfusion system and/or a lid as described above. In those embodiments, wherein the cell culture device comprises both perfusion system and lid, there are two ways to access the liquid inside the culture device: from the top or through the microfluidic channels. Naturally, removing the lid enables access from the top (i.e. the open side of the cell culture chamber). As mentioned before, this allows using a manual pipette or a robotic dispenser for exchanging the fluid inside. Furthermore, an open culture device allows performing confocal microscopy using dipping lenses.

Access through the microfluidic channels, on the other hand, permits continuous fluid exchange inside the cell culture chamber even when the cell culture chamber is closed by a lid. If the device is connected to a pump, this fluid exchange can be automated, including pre-programmed concentration ramps, for instance, for cell reaction triggering compounds (stimulants). The fluid level inside the cell culture chamber can be adjusted by adapting the flow parameters. In addition, cells can grow under defined flow conditions (shear stress).

In exemplary embodiments of the cell culture device, the bottom plate may have one or more electrodes attached thereto, preferably within the cell culture chamber(s). The one or more electrodes may serve a variety of purposes: They may be provided to apply electric potentials to stimulate the one or more cells, they may be provided for measuring pH, impedance, temperature or conductivity, for instance. They may also be provided to heat the cell culture chamber. In the latter case, they may also be provided on a sidewall and/or outside the cell culture chamber.

In those embodiments, the one or more electrodes may be made of metal, such as gold, platinum or ruthenium, or graphite, indium tungsten oxide (ITO) or conductive ethoxycarbonyl-based organic material, for instance. ITO electrodes are advantageous in that they are transparent to visible light. ITO, gold and ruthenium electrodes would be preferred for the application of potentials, such as for stimulation of the one or more cells. Ruthenium electrodes may be included for oxygen sensing.

In further embodiments of the present invention, the bottom plate can be a silicon wafer. The silicon wafer may be a blank silicon wafer or may comprise one or more complementary metal oxide semiconductors (CMOS). The silicon wafer may further comprise one or more electrodes supported by the one or more complementary metal oxide semiconductors (CMOS).

In a further embodiment, the bottom supporting foil of the force sensor array may be laminated to a CMOS sensor array or other electrode arrangement. In those embodiments, it is advantageous for the bottom supporting foil of the force sensor to be very thin, for instance about 75 micrometers, preferably 50 micrometers, or less.

According to a further aspect, the present invention provides a force sensor array, in particular for use as part of the cell culture device as described herein, wherein the force sensor array comprises a plurality of discrete observation spots, which are spaced apart from one another, each observation spot comprising a plurality of flexible microcolumns, wherein each observation spot has a unique label disposed adjacent to it allowing identifying and/or locating each observation spot. Thus, the force sensor array comprises an intrinsic guidance system.

Each label may comprise at least one of a symbol, letter, number, ornament or combination thereof.

The observation spots are preferably arranged in an array, such as a matrix of rows and columns, each label identifying row and column of the observation spot where the associated observation spot is located, or more generally the location in the array.

Further preferably, a plurality of matrices (or arrays) of observation spots is arranged in an observation field, wherein each label identifies the location both within the observation field and the observation spot matrix. In those embodiments, preferably, each label comprises four variables, a first variable indicating the column of the observation spot matrix, a second variable indicating the row of the observation spot matrix, a third variable indicating the column of the observation field and a fourth variable indicating the row of the observation field, or more generally the respective locations within the array if the array is not in rows and columns. Expressed more generally, each observation spot is associated with a label that comprises a number of variables, wherein the labels of different observation spots differ in at least one variable.

Such an intrinsic guidance system allows better orientation while performing an experiment as well as re-locating a particular cell formation. This is especially useful for subsequent staining or fixation procedures. The embodiment of the intrinsic guidance system presented above has a hierarchic structure dividing the complete sensor field (force sensor array) into observation fields and provides a plurality of levels.

In an exemplary embodiment, the complete force sensor array of a single cell culture chamber has 6×9 observations fields, which are each subdivided into 12×8 observation spots thus making 5184 discrete sensor arrays with unique labels. With a design of 12×12 microcolumns (force sensors) within each observation spot (enough to accommodate a few single cells) such a force sensor array comprises almost 750 000 individual microcolumns (force sensors) in an area of 13×12.5 mm². More generally, the complete force sensor can have 2 to 50×2 to 50 observation fields, for instance 3 to 30×3 to 30 observation fields, preferably with each observation field being subdivided into 2 to 50×2 to 50 observation spots, for instance 4 to 40×4 to 40 observation spots. In an alternative exemplary embodiment, the complete force sensor array of a single cell culture chamber has 4×4 observations fields, which are each subdivided into 12×8 observation spots.

The intrinsic guidance system for the force sensor array described herein greatly facilitates principle orientation for the user while browsing the sensor array with a microscope at higher magnification (e.g. 10× lens and higher). It also simplifies documentation and correlation of data from different experiments for the same set of cells, which is especially interesting for long-term experiments. Labelling each observation spot uniquely allows identifying the exact position even without a controlled motorized stage. Furthermore, it is invaluable for repeated investigations of the same observation spot even if the device is removed from the microscope stage.

According to a further aspect, a force sensor array is provided, in particular for use as part of the cell culture device according to the present invention, wherein the force sensor array comprises a plurality of discrete observation spots, which are spaced apart from one another, wherein each observation spot comprises a plurality of flexible microcolumns, and wherein the observation spots are arranged in a matrix of rows and columns, or more generally in a predetermined pattern, and wherein a plurality of matrices of observation spots are arranged in an observation field, the force sensor array further comprising position markers for indicating a position of the observation spots within the array.

This latter embodiment does not necessarily comprise a refined hierarchy, as described for the previous embodiment, but allows for principal orientation in the force sensor array.

According to a further aspect, the present invention provides a method for culturing cells and measuring mechanical forces exerted by one or more cells, comprising:
(i) placing one or more cells on a force sensor array of a cell culture device, preferably the force sensor array preferably of the cell culture device of the present invention as described herein,
(ii) culturing the one or more cells, and
(iii) detecting, preferably measuring, a force exerted by the one or more cells.

Placing the one or more cells on the force sensor array generally encompasses placing the one or more cells on an observation spot of the force sensor array.

For instance, cells are cultivated inside the cell culture chamber for a few days or at least several hours, which is the time needed by cells to establish focal adhesion points with a suitable surface. Focal adhesions link the cytoskeleton to the external cell matrix with complex protein structures. This is preferably simulated by coating the here described microcolumns with proteins like laminine, fibronectine or collagen, as mentioned above.

Step (ii) of the method according the present invention may further comprise exchanging culture medium, for instance via the perfusion system. Step (ii) of the method according the present invention may further comprise a continuous exchange of culture medium, for instance via the perfusion system, for instance in an automated fashion.

The method according to the present invention may further comprise a step of applying a chemical and/or mechanical and/or electric stimulus (to the one or more adhered cells) after step (ii) and before and/or during step (iii).

As explained before, a cell reacting to the stimulus changes its shape, thus rearranging the position of the microcolumns (force sensors) within the force sensor array. Video footage of this process gives detailed information of the process. Each microcolumn deflection provides a value for the exerted forces. Subsequent analysis of the data gained from the video footage allows drawing a two-dimensional vector field illustrating the process.

In particular in combination with fluorescent labeling of particular sub-cellular structures, e.g. actine fibres, the sensor deflection can be correlated with processes that happen inside the cell. In a further embodiment, the method according to the present invention comprises a step of fluorescent labeling one or more cells. The fluorescent label may be attached to actine fibers, cell membrane and/or cell nucleus, for instance. Such an embodiment preferably further comprises measuring a force exerted by the one or more fluorescently labelled cells, in particular a deflection of the microcolumns, by microscopy, for instance standard microscopy or phase contrast microscopy or differential interference contrast microscopy. Such an embodiment preferably further comprises detecting fluorescence generated by the fluorescent labeled cells. Such an embodiment further preferably comprises correlating the detected fluorescence with the measured force.

A mechanical stimulus may be provided by shear conditions. Shear conditions can be controlled by flowing liquid through the cell culture chamber, in particular making use of the perfusion system according to the present invention, as described above. Step (ii) and/or step (iii) may comprise application of shear conditions.

Typically, step (iii) comprises detecting a deflection of one or more microcolumns by optical means. The detection may comprise detection of transmitted light, fluorescent light and any other conceivable means, for instance by standard microscopy or phase contrast microscopy or differential interference contrast microscopy. As described before, detecting a deflection generally comprises detecting a first position of a microcolumn and detecting a second position of the same microcolumn and determining a force vector based on the first and second positions.

In an exemplary embodiment, additional cells may be cultured adjacent to the force sensor array.

Step (i) may comprise placing one or more cells of a first type in a first position on the force sensor array, such as a first observation spot, and placing one or more cells of a second type in a second position on the force sensor array, such as a second observation spot, wherein the first type of cells is different from the second type of cells and the first position is different from the second position.

Step (iii) may further comprise adding a substance to the one or more cells, such as a chemical stimulant, for screening whether said substance causes the one or more cells to exert a mechanical force.

According to a further aspect, the present invention provides use of a device according to any aspect described herein for both culturing cells and subsequent measuring mechanical forces exerted by one or more of the cultured cells.

In the following, exemplary embodiments of the present invention will be described with reference to the Figures wherein

FIG. 1 illustrates the general principle of force sensor arrays,

FIG. 2 shows a view onto the top of an embodiment of a cell culture device according to the present invention comprising two cell culture chambers,

FIG. 3 shows a view onto the bottom of the cell culture device shown in FIG. 2,

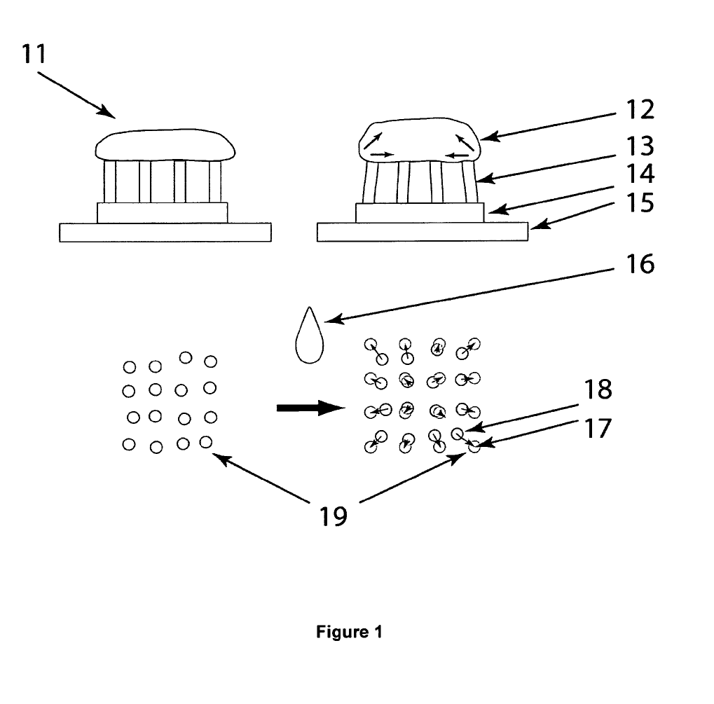

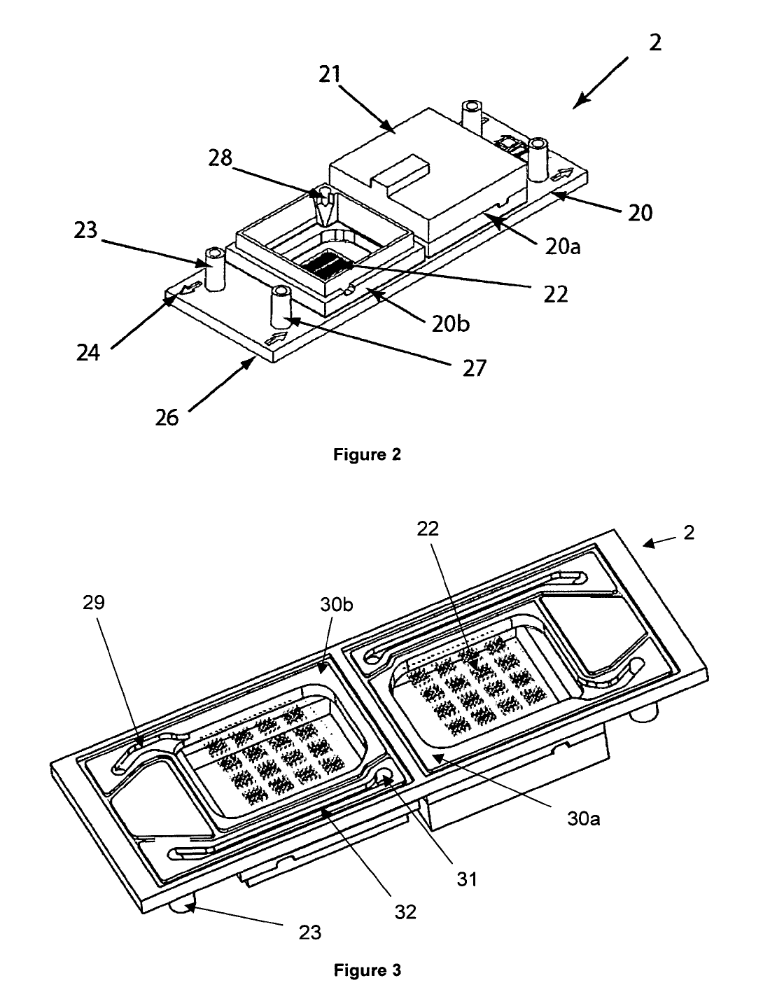

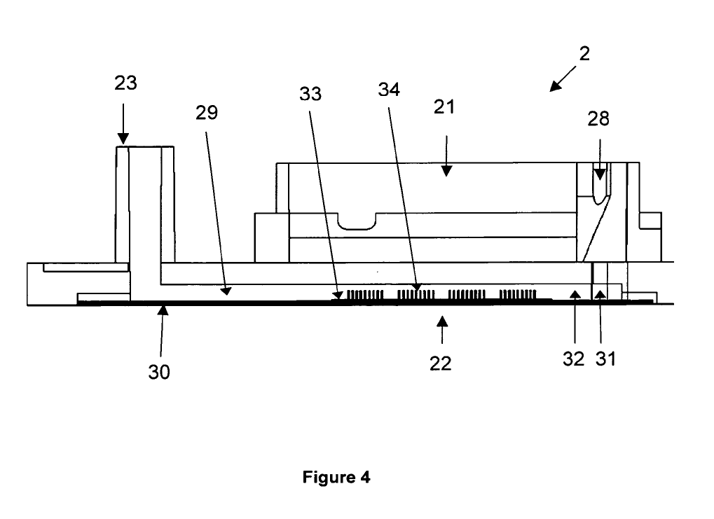

FIG. 4 shows a cross-section of the cell culture device shown in FIGS. 2 and 3,

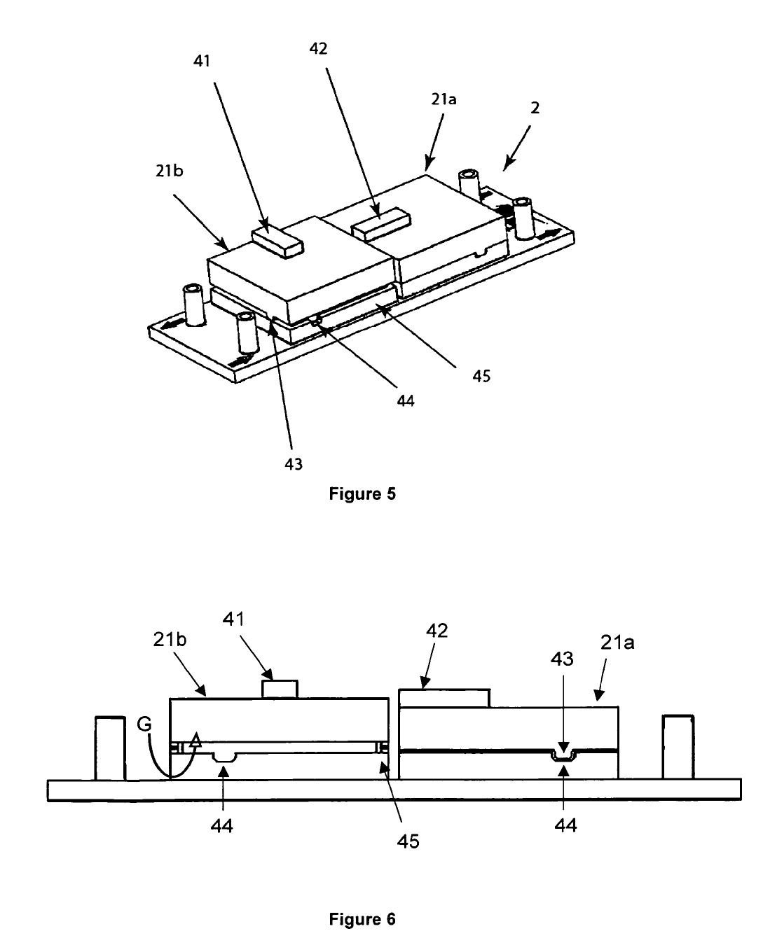

FIGS. 5 and 6 illustrate structure and function of the lids, and

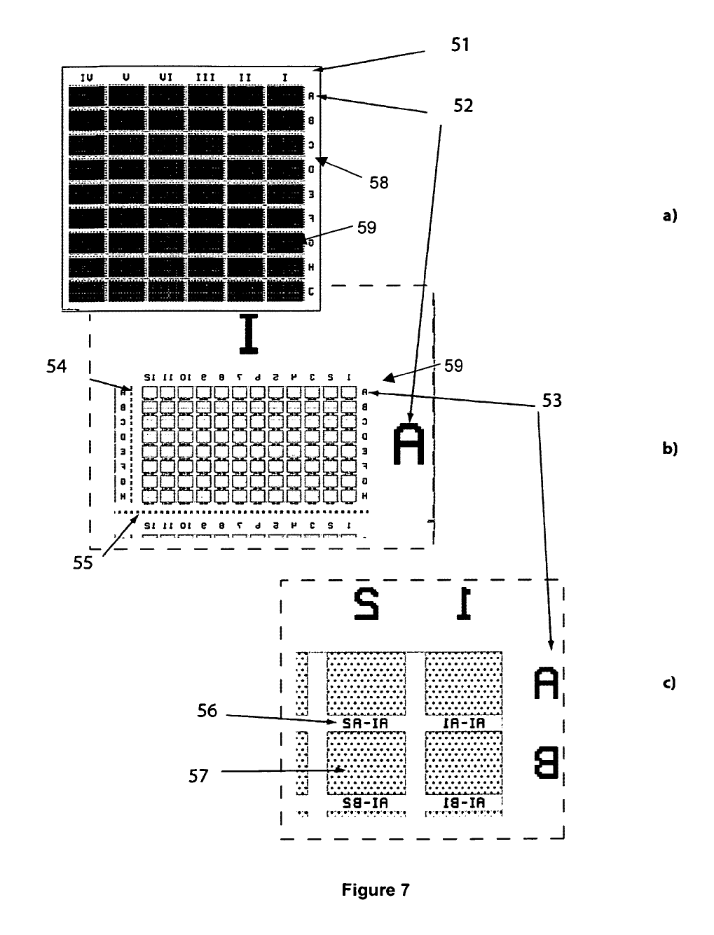

FIG. 7 illustrates an embodiment of the present invention comprising an intrinsic guidance system, and

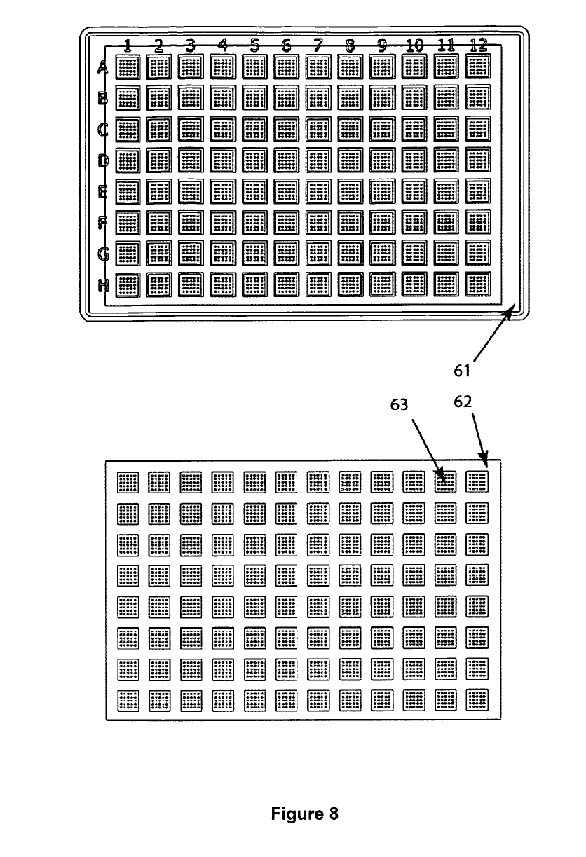

FIG. 8 shows a further exemplary embodiment of the present invention.

FIG. 2 shows an embodiment of a cell culture device (2) according to the present invention comprising a force sensor array (22) for measuring cell contraction, for instance. It comprises a main frame (20) that includes two cell culture chambers (20a, 20b) enclosed by sidewalls. One of the cell culture chambers (20b) is depicted in open form, the other one is depicted covered by a removable lid (21). The main frame has an opening in the bottom where a bottom plate is disposed. The bottom plate of the cell culture device is transparent and carries the force sensor array (22). The cell culture device (2) further comprises a perfusion system, of which in FIG. 2, the first and second connectors (“Outlet” 23, “Inlet” 27) and a fill level overflow opening (28) are visible. The fill level overflow opening (28) pulls in any excess liquid, which is guided out through a microfluidic channel (not visible) and connector “Outlet” (23). The liquid level inside the cell culture chambers can be regulated, for instance, by applying negative pressure to the (second) connectors “Outlet” (23). The chamber(s) can be filled either by direct pipetting through the opening on the top or through the (first) connector(s) “Inlet” (27). The connectors (23, 27) are suitable to connect to elastic tubing, for example. Indicators (24, 26) associated with the connectors (23, 27) help the user to correctly connect the chamber perfusion, in particular identify which connector is in fluid communication with the fill level overflow opening (28).

FIG. 3 shows the bottom side of the cell culture device (2) depicted in FIG. 2. The force sensor arrays (22) are clearly visible through the bottom plates (30a, 30b). The transparent bottom plates are only just visible (30a, 30b). The bottom plates (30a, 30b) fit exactly into holding structures (recesses) provided on the bottom side of the main frame (20), such that the bottom surface of the main frame (20) and the bottom plates (30a, 30b) substantially form one even surface. On the bottom surface of the main frame (20), two microfluidic channels are provided for each cell culture chamber. They are sealed on one side by the bottom plates (30a, 30b). A first microfluidic channel (29) is connected to the connector “Inlet” (not visible) and provides fluid communication between a lower part of the cell culture chamber and the connector (“Inlet”), thus allowing to fill the cell culture chamber from the outside. A second microfluidic channel (32) provides fluid communication between the connector “Outlet” (23) and the fill level overflow opening, of which the bottom part (31) is visible.

FIG. 4 shows a cross-section (not to scale) through a part of the cell culture device (2) already shown in FIGS. 2 and 3. Like reference numerals refer to like components. The cross-section nicely illustrates the recess in the main frame (20) providing a holding structure for the bottom plates (30). It can further be seen how the microfluidic channels (29, 32) are sealed on one side by the bottom plate (30). Furthermore, the components of the force sensor array (22) are visible: a plurality of microcolumns (34) and bottom supporting foil (33), which is formed as one piece from an elastomeric material, such as PDMS. The bottom plate (30) is glued or welded to the main frame (20). The bottom supporting foil (33) of the force sensor array is bonded or glued to the bottom plate (30).

FIGS. 5 and 6 illustrate structure and functioning of exemplary embodiments of the lids. FIG. 5 shows a view onto top/side of the cell culture device (2) whereas FIG. 6 shows a cross-section of the same device (2). The lids (21a, 21b) take the form of a kind of rectangular box with a bottom and sidewalls. They are used such that the sidewalls face towards the sidewalls of the cell culture device (2). On a rim of the sidewall designed to rest against a rim (45) around the sidewalls of the cell culture chambers, the lids (21a, 21b) are each provided with a protrusion (43). The rims (45) around the sidewalls of the cell culture chambers are each provided with an indentation or recess (44) configured to receive the protrusion (43). For the cell culture chamber on the right-hand side of FIG. 5, the lid (21a) is positioned in a first position on the cell culture chamber such that the protrusion (43) on the lid (21a) comes to rest within the recess (44) provided in the rim (45) around the side walls of the cell culture chamber. This represents a first closing modus wherein the opening of the cell culture chamber is sealed by the lid. When the cell culture chamber is filled with liquid, no gas exchange with the environment can take place. The cell culture chamber on the left-hand side of FIG. 5 shows the alternative position, providing the second closing modus: The lid (21a) is positioned on the cell culture chamber such that the protrusion (43) on the lid is in a different position from the recess (44) in the rim (45) around the side walls. The lid (21a) is turned by 90 degrees compared to lid (21b.) In that position, the protrusion (43) comes to rest on the rim (45) around the sidewalls and provides a gap between the side walls of the cell culture chamber and the lid (21a), thus allowing exchange of gas G between the cell culture chamber and the environment.

The lids (21a, 21b) each comprise a position indicator (41, 42) to facilitate identifying in which position to apply the lid to the cell culture chamber for a desired closing position. The chamber is closed when the position indicator (42) shows toward the device centre. Gas exchange is possible in any other position of the position indicators (41, 42). The possibility for gas exchange becomes particularly apparent in FIG. 6, in which like reference numerals refer to like features as in FIG. 5. In summary, the gas exchange between the inside of the cell culture chambers and the environment can be regulated by using the depicted lids, and in particular spacer constructions provided thereon.

FIG. 7 shows an intrinsic guidance system provided with the force sensor array, which has multiple levels. On the top-level (51) (s. FIG. 7a), letters along the vertical axis and Roman numbers along the horizontal axis divide the complete sensor field (58) into a rectangular matrix of observation fields (59). The observation fields (59) are separated from one another by lines made of the corresponding letter or number, respectively (54, 55). Furthermore, each observation field (59) is subdivided into discrete observation spots (57) (see FIG. 7c)). On this level, the numbering along the vertical axis comprises letters, and the numbering along the horizontal axis is provided by Arabic numbers. By combining the numbering from both levels, each observation spot (57) has a unique label (56). Each observation spot (57) has a sub-sub-array of microcolumns (force sensors). This intrinsic guidance system allows to exactly identify and re-locate each observation spot (57).

FIG. 8 shows a further embodiment of a cell culture device according to the present invention. This embodiment is configured as a 96-well microtiter plate with transparent bottom plate (61) and a force sensor array for each well (63).

1. Cell culture device for culturing cells and measuring mechanical forces exerted by one or more cells, comprising:
a cell culture chamber, comprising a bottom plate and a main frame comprising side walls to enclose a volume of the culture chamber together with the bottom plate,
the main frame with side walls being fabricated as one piece,
the cell culture chamber being open on a side of the cell culture chamber opposite the bottom plate,
the cell culture chamber further comprising a force sensor array disposed on top of the bottom plate within the cell culture chamber, the force sensor array comprising an array of flexible microcolumns configured to provide a site of adhesion for one or more cells, each microcolumn being configured to be deflected from an original position to a deflected position in response to a mechanical force exerted by the one or more cells.

2. Cell culture device according to claim 1, wherein at least a part of the bottom plate is provided by a transparent plate.

3. Cell culture device according to claim 2, wherein the transparent plate is a glass plate.

4. Cell culture device according to claim 1, wherein the main frame comprises a holding structure for holding the bottom plate.

5. Cell culture device according to claim 1, wherein the bottom plate is made from a rigid material.

6. Cell culture device according to claim 1, wherein the bottom plate is an integral part of the main frame.

7. Cell culture device according to claim 1, wherein the main frame is made from a polymeric material.

8-16. (canceled)

17. Cell culture device according to claim 1, further comprising a perfusion system for delivering liquid to and removing liquid from the cell culture chamber.

18. Cell culture device according to claim 17, wherein the perfusion system forms an integral part of the main frame.

19. (canceled)

20. Cell culture device according to claim 17, the perfusion system comprising a first microfluidic channel providing fluidic communication between a first connector located on the main frame outside the cell culture chamber and the cell culture chamber for delivering fluid into the cell culture chamber and a second, separate microfluidic channel providing fluidic communication between a second connector located on the main frame outside the cell culture chamber and the cell culture chamber for removing fluid from the cell culture chamber.

21-28. (canceled)

29. Cell culture device according to claim 1, wherein the force sensor array is made of a silicone elastomer, a mixture of silicone elastomers or a mixture of a silicone elastomer and a silicon fluid.

30-34. (canceled)

35. Cell culture device according to claim 1, comprising two or more cell culture chambers.

36-38. (canceled)

39. Cell culture device according to claim 2, wherein the transparent plate has one or more electrodes attached.

40-44. (canceled)

45. Force sensor array for use as part of the cell culture device according to claim 1, the force sensor array comprising a plurality of discrete observation spots, which are spaced apart from one another, each observation spot comprising a plurality of flexible microcolumns, wherein each observation spot has a unique label disposed adjacent to it allowing identifying and/or locating each observation spot.

46-47. (canceled)

48. Force sensor array for use as part of the cell culture device according to claim 1, the force sensor array comprising a plurality of discrete observation spots, which are spaced apart from one another, each observation spot comprising a plurality of flexible microcolumns, wherein the observation spots are arranged in a matrix of rows and columns, and wherein a plurality of matrices of observation spots are arranged in an observation field, the force sensor array further comprising position markers for indicating a position on the array.

49. Force sensor array according to claim 45, wherein each label comprises at least one of a symbol, letter, number, ornament or combination thereof.

50-52. (canceled)

53. Method for culturing cells and measuring mechanical forces exerted by one or more cells, comprising:
(i) placing one or more cells on the force sensor array of the cell culture device according to claim 1,
(ii) culturing the one or more cells,
and
(iii) measuring a force exerted by the one or more cells.

54-58. (canceled)

59. Method according to claim 53, wherein step (iii) further comprises adding a substance to the one or more cells for screening whether said substance causes the one or more cells to exert a mechanical force.

60. Use of a device according to claim 1 for both culturing cells and subsequent measuring mechanical forces exerted by one or more of the cultured cells.
